(19) United States
(12) Patent Application Publication
(10) Pub. No.: US 2026/0345365 A1
(43) Pub. Date: Nov. 5, 2026

(54) DEVICE FOR LIMITING AND/OR DAMPING A FOOT MOVEMENT VIA THE ANKLE JOINT, AND SHOE

(71) Applicant: BETTERGUARDS TECHNOLOGY GMBH, Berlin (DE)

(72) Inventor: Vinzenz BICHLER, Berlin (DE)

(73) Assignee: BETTERGUARDS TECHNOLOGY GMBH, Berlin (DE)

(21) Appl. No.: 18/881,284

(22) PCT Filed: Jun. 30, 2023
(86) PCT No.: PCT/EP2023/068119

§ 371 Date: May 22, 2026

(30) Foreign Application Priority Data
Jul. 5, 2022 (DE) 102022116790.9

(51) Int. Cl. A61F 5/01

(52) U.S. Cl. CPC A61F 5/0118

(57) ABSTRACT

The present invention relates to a device (1) for limiting and/or damping a foot movement via the ankle joint, comprising: a support assembly (2) for support on a lower limb (112) and/or proximally on the ankle-joint malleolus (114); a holding assembly (3) for holding on a foot (110), preferably on a sole region of a foot (110); and at least one connection element (4), which extends between the support assembly (2) and the holding assembly (3) and is connected to each, further comprising a tightening assembly (5) for tightening the connection element (4), which tightening assembly is designed such that the connection element (4) between the holding assembly (3) and the support assembly (2) can be changed between a loosened state and a tightened state. The invention further relates to a show (100) for limiting and/or damping a foot movement via the ankle joint.

### TECHNICAL FIELD

The present invention relates to a device for limiting and/or damping a foot movement via the ankle joint and a shoe for limiting and/or damping a foot movement via the ankle joint.

### BACKGROUND

It is known in the art to stabilise the movement of the ankle joint by means of devices for limiting or damping foot movements in order to prevent trauma resulting from an ankle twist, i.e. a movement of the ankle joint across at least one of its ankle joint axes in a non-physiological range. The most common form of ankle twist trauma is an ankle sprain in the form of a stretch or rupture due to inversion. Inversion movements across an ankle joint axis result in a change in the distance between the foot and lower leg due to the increasing inversion angle. Exceeding a certain inversion angle or a certain inversion speed or inversion acceleration, respectively, may cause injuries to the ankle ligaments or ruptures.

Devices are known in the art to prevent this that allow a certain range of movement and completely prevent movement about the ankle joint axis in excess of a certain threshold angle of movement. On the one hand, relatively rigid orthoses are known in the art for this purpose, which focus on inhibiting movement using splints or splint plates.

Furthermore, devices or shoes are known in the art that allow movement of the ankle joint up to a certain threshold angle of movement, and movement in excess of this threshold angle is completely prevented based on the device's or shoe's construction. A corresponding device is known, for example, from EP 2 717 809 A2.

Furthermore, devices are known in the art that, when worn, always allow for a minimum of movement but block during dangerous movements. DE 10 2014 107 335 A1 shows a device for adaptively limiting inversion or supination movements across the ankle joint. In order to prevent the upper part of the orthosis from shifting in the direction of the foot when force is exerted by the foot, the ankle joint orthosis shown therein requires a pressure-stable beam located on the medial side of the ankle joint to support the upper area, which extends between the upper area of the orthosis and the lower area of the orthosis and must be firmly connected to both areas. The beam must be designed in such a way that it redirects the tensile forces introduced into the upper area via the pull-out device on the lateral side back into the lower area as compressive forces on the medial side without deforming or buckling. This ensures that its position is secured in the distal direction. However, the beam on the medial side of the foot still restricts freedom of movement of the ankle joint.

It is also known in the art to provide a fixture for securing the upper part to a lower leg instead of providing securing between the upper and lower part on the upper part. In this case, the upper part rests on the ankle from above due to the fixture. Due to the support and fixture, these devices are associated with high weight as well as a relatively rigid structure and poor wearing comfort.

Newer devices and shoes, respectively, are also known in the art providing a support assembly resting on the lower leg secured by means of a circumferential strap that is deflected in an eyelet, tightened and then secured. This support assembly is connected to a securing assembly for securing to a foot via a damping element for damping a relative movement. Such devices derive from DE 10 2018 124 932 A1 or DE 10 2017 109 877 A1.

However, the above-mentioned devices must be adapted to the dimensions of the wearer's lower leg in order to be fully functional. If individual components of the device are not in the intended position on the wearer's body when worn, this may reduce the protective effect of the device. In extreme cases of unsuitable positioning, the protective function may even be rendered obsolete completely.

Up to now, this problem has been solved by providing limiting devices in different sizes. However, even at the same foot or shoe size, the girth of the lower leg, ankle and foot varies considerably from one person to the next. Accordingly, for wearers with comparatively thin feet, if the device is placed on a “standard foot” with larger dimensions of the foot area, such as ankle circumference or ankle cross-section (area), lower leg circumference or lower leg cross-section (area), as well as height and thickness of the mid-foot and forefoot, the device may sit (too loosely) on the foot when worn so that the device, in particular the components of the device intended to limit or dampen movement of the foot, such as tensile-stiff areas and straps, will first align and tighten before any protective effect may is effected, when twisting occurs. Accordingly, it is possible that in the case of comparatively slender feet with a comparatively small ankle circumference, the protective effect in respect to an inversion angle and/or supination angle, for example, may not be achieved as intended, but only once an undesired degree of deflection from the neutral position has occurred, already. In the case of feet with comparatively large dimensions or comparatively thick feet with a comparatively large ankle circumference, the components of the device responsible for the protective function, in particular tensile-stiff straps, may already be under tension when the foot is in a neutral position due to the substantial thickness of the foot or may already cause their limiting and/or damping effect in relation to, for example, an inversion angle and/or supination angle, so that the mobility of the foot about the ankle joint may already be restricted during physiological movements from which no injury is generally to be expected. To cater for the above characteristics, the device can be customised for each individual wearer. However, due to the associated need for bespoke designs, custom-made devices are comparatively expensive, which results in high acquisition costs and excessive effort, making such solutions inaccessible to the majority of wearers.

### SUMMARY OF THE INVENTION

Starting from the prior art, it is one object of the present invention to provide an improved device for limiting and/or damping a foot movement via the ankle joint as well as a corresponding shoe.

The object is solved by a device for limiting and/or damping a foot movement via the ankle joint with the features of claim 1. Advantageous further embodiments result from the dependent claims, the description and the figures.

Accordingly, a device is proposed for limiting and/or damping a foot movement via the ankle joint, comprising a support assembly for support on a lower limb and/or proximally on the ankle-joint malleolus; a holding assembly for holding on a foot, preferably on a sole area of a foot; and at least one connection element, which extends between the support assembly and the holding assembly and is connected to each.

The connection element is a force-transmitting connection between the support assembly and the holding assembly, through which forces can be transmitted between the holding assembly and the support assembly. Preferably, the connection element is a connection strap.

The device further comprises a tightening assembly for tightening the connection element, wherein the tightening assembly is designed such that the connection element between the holding assembly and the support assembly can be switched from a loosened state to a tightened state and vice versa.

In the present context, the term “tightening” is understood to mean creating tension or inducing tension. Accordingly, the “tightened state” corresponds to a state in which the connection element is in a pre-tensioned state. The term “loose” or “loosened state” is also defined as a state in which the connection element is loose, i.e. without being subjected to tension. In the loosened state, the connection element is displaceable at least at least one of a connection to the support assembly, preferably at least one of the first connection and/or the second connection, and a holding assembly connection. Accordingly, the tightening assembly allows for changing the length of the connection element between its connection to the support assembly and its holding assembly connection to the holding assembly, preferably between the first connection and the second. In the loosened state, the length of the connection element between its connection to the support assembly and its holding assembly connection to the holding assembly, preferably between the first connection and the second connection, can be extended in such a way that fitting the device in its intended position on the foot may be enabled and/or facilitated. By “tightening” at the connection element, preferably at or in the area of a connection to the support assembly and/or a free end of the connection element, particularly preferably at or in the area of the first connection or the second connection, shortening the length of the connection element between the support assembly and the holding assembly is effected, preferably between the first connection and the second connection. If the connection element is initially loose after the device has been attached to the foot in the loosened state, tightening the connection element or the associated shortening of the length of the connection element between the support assembly and the holding assembly, preferably between the first connection and the second connection, effects the tightening. In other words, the tightening assembly allows the connection element between the support assembly and the holding assembly, preferably between the first connection and the second connection, to be conveyed into a “state of tension”, i.e. tension, preferably tension in a predetermined range or a predetermined amount of tension, can be applied to the connection element. As a result, the connection element is tightened between the support assembly and the holding assembly, whereas it is preferably tightened, or in any case pre-tensioned, between the first connection and the holding assembly connection and is tightened, or in any case pre-tensioned between the holding assembly connection and the second connection. In other words, in the tightened state, the connection element is tensioned or in any case pre-tensioned between the support assembly and the holding assembly.

The fact that the device comprises a tightening assembly for tightening the connection element, which is configured in such a way that the connection element can be switched between a loosened state and a tightened state between the holding assembly and the support assembly, allows for adapting the device to the dimensions, in particular the ankle circumference, of the wearer's foot, and in particular the connection element, independently and separately from other components of the device or from their fastening to the leg and foot of the wearer. Accordingly, the connection element can be conveyed into a predetermined state, in particular a predetermined pre-tension state, in an intended position of the foot, preferably a neutral position of the foot. Thus, the protective effect of the device can be made available to a large number of wearers or potentially even any wearer in a predetermined range, i.e. neither too early or too late and/or too much or too little, in the case of feet with different dimensions and in particular with different ankle circumferences. In particular, contrary to the conditions in conventional devices known in the art, this is possible without other components of the device having to be fixed to the lower leg at a distance from the ankle, for example by tightening a sling or loop in such a way that slipping of the support assembly from is sought to be prevented by means of the pressing force and the associated adhesive friction, as is the case with known, conventional devices. This approach will cause the foot to be “constricted”, or at the very least will result in poor wearing comfort, especially with the blood circulation to and from the foot also potentially be negatively affected. In contrast thereto, the device presently proposed does not require tightening of any components in a non-physiological manner or arranging them at a location other than the intended location in the ankle area in order to achieve the desired protective effect within the desired range of movement. In other words, the attainable tightening or pre-tensioning of the connection element is decoupled from attaching or fastening in particular of the support assembly of the device to the foot above the ankle or on the proximal side of the wearer's ankle.

The neutral position of the foot preferably corresponds to the zero position of the foot with regard to supination/pronation, inversion/eversion and/or plantar extension/dorsal extension or with regard to the respective angular position thereof.

The terms lateral, medial, posterior, anterior, proximal, distal, dorsal and plantar are to be understood in such a way that they correspond to the anatomical directional designations when the device or shoe is correctly fitted to a foot.

The term “lateral side” therefore presently refers to an outer side of the device. In this context, the lateral side of the device corresponds to a lateral side of a human foot or a lower human limb when the shoe is worn. The “lateral side” is located on the side or facing away from the centre of the wearer's body when the device is worn. In other words, the term “lateral side” comprises a lateral side of the human body in the sense of (topographical) anatomy.

Correspondingly, the term “medial side” presently comprises an inner side of the device. The medial side of the device corresponds to the medial side of a human foot or a lower human limb when the device is worn. The “medial side” is oriented towards the centre of the body or is arranged centrally when the device is worn. In other words, the term “medial side” comprises a medial side of the human body in the sense of (topographical) anatomy.

Furthermore, the term “dorsal” corresponds to a top side of the foot, the term “plantar” corresponds to a bottom side of the foot, the term “proximal” corresponds to oriented or located towards the centre of the body, the term “distal” corresponds to oriented away or located away from the centre of the body.

A “heel area” comprises an area of the device or a shoe in which the heel of a foot is located when the device is worn. Consequently, the heel area corresponds to a rear or posterior side of the device in the sense of the human body's (topographical) anatomy. Consequently, the heel area of the shoe is located opposite an anterior side.

In the case in hand, the term “ankle joint” comprises the upper and lower parts of the ankle joint and, accordingly, the axis of movement of the upper part of the ankle joint, which essentially allows for plantar flexion and dorsal extension of the foot, as well as the axis of movement of the lower ankle joint, which essentially allows for inversion and eversion as well as supination, adduction and plantar flexion, abduction and dorsal extension.

In the present context, the term “ankle” is also used to refer to the elements of the joint socket, the malleolus fork of the upper ankle joint. Consequently, the term “ankle” in the present case includes the outer part of the ankle, which is formed by the lateral malleolus, and the inner part of the ankle, which is formed by the medial malleolus. Due to the design of the malleolus fork as a joint socket, the ankle has a larger cross-sectional area in relation to section of the lower leg located above and thus proximally in relation to the lower leg and in respect of the proximal-distal direction. Consequently, the circumference of the ankle is larger compared to the proximally adjoining section.

According to a further preferred embodiment, the connection element is connected to the support assembly via a first connection and a second connection and is connected to a holding assembly connection at the holding assembly between the first and second connection. In other words, the connection element preferably extends from the first connection to the holding assembly connection, and then on to the second connection. A further advantageous embodiment has turned out to be a configuration in which the connections are positioned relative to the connection element in such a way that a restraining force generated in the connection element as a result of a foot movement via the ankle joint is divided into a laterally-proximally acting component and a medially-proximally acting component at the support assembly. This in particular allows for the restraining force generated by the connection element on both sides of the foot or lower leg of a person wearing the foot movement limiting device to be transferred to the support assembly, so that opposite the connection element support of the support assembly is effected on the lower leg or ankle in the form of a linear load that is effective from the first connection to the second connection posteriorly around the lower leg or around the proximal region of the ankle. In other words, support is generated by tightening the support assembly on both sides, allowing the support assembly to thus support itself on the ankle essentially without being displaced. To this end, the support assembly preferably comprises at least one area with tensile rigidity, preferably extending from the first connection posteriorly around the circumferential direction of the lower leg to the second connection.

The connection element is preferably an elongated connection element, it preferably comprises a strap or is designed as a strap. In other words, the connection element is a connection strap, i.e. a force-transmitting strap, through which forces can be transmitted between the support assembly and the holding assembly. The connection element preferably has tensile-stiff properties at least in the longitudinal direction of the connection element; in any case it at least comprises a material that is tensile-stiff at least in the longitudinal direction.

In the present context, the term “strap” is understood to mean an elongated element which, on the one hand, is characterised by tensile stiffness and in any case is therefore capable of transmitting tensile forces and, on the other hand, is bendable or flexible transversely to the longitudinal direction of the strap, so that it can be applied to a corresponding structure transversely in respect to longitudinal direction. In addition, a strap can be easily deflected. In the case in hand, the term strap generally refers to an elongated, flexible, optionally elastic element, which may be provided in the form of a single fibres, a strand of fibres, a wire, a cord, a rope, a textile fabric with limited width and fixed selvedges on both sides or the like.

In this context, “tensile strength” applies to any material that can be used to transmit tensile forces. Tensile-stiff materials within the meaning of this document may display a certain initial elongation capacity and preferably stiffen after a certain elongation threshold limit or yield strength threshold value has been exceeded in such a way that the elongation capacity is then greatly reduced compared to the initial elongation capacity or yield strength. In the case in hand, a textile braid made of threads of a tensile-stiff material may for example be used as a tensile-stiff strap, so that when tensile load is applied to the textile braid, the threads of the textile braid initially are subjected to increasing alignment in the longitudinal direction of the textile braid, so that high initial elongation capacity is initially provided due to the alignment of the threads and, subsequent to the alignment of the threads, the strap essentially exhibits a tensile-stiff structure in the direction of the force flow. In the alternative, the material may also exhibit essentially immediate tensile-stiff properties.

The fact that the connection element is designed to exhibit tensile stiffness in the longitudinal direction is thus to be understood to mean that the connection element is capable of transmitting tensile forces in the longitudinal direction when in a tightened or stretched state, essentially without experiencing elongation when subjected to tensile forces or in any case on only experiencing slight elongation in such an event, similar to a seat belt in vehicles.

According to a preferred embodiment, the connection element extends between the first, preferably lateral, connection and the second, preferably medial, connection on a front side, i.e. an anterior side of the device.

Preferably, the connection element is fastened to the holding assembly connection via an articulated holder. In other words, the holding assembly connection is preferably an articulated holder or is provided as such.

In this context, an articulated holder or “held in an articulated manner” means in particular that the connection element at the holding assembly connection, i.e. the connection point to the holding assembly, has at least a certain degree of freedom in relation to the holding assembly. Preferably, the connection element is pivotable about a pivot axis, preferably in any case in a predetermined angular range.

According to a further preferred embodiment, the support assembly comprises the tightening assembly for tightening the connection element. This in particular enables the wearer of the device to tighten or re-tighten the connection element also while wearing the device, for example when the device is worn under a shoe or as part of a shoe.

The tightening assembly is preferably configured to tighten the connection element between the first connection and the second connection.

Furthermore, the first connection may optionally be a lateral connection, i.e. a connection arranged on the lateral side of the device, and/or the second connection can be a medial connection, i.e. a connection arranged on the lateral side of the device.

The connection element may optionally be fastened to the holding assembly connection so as to be displaceable relative to the holding assembly. In other words, the holding assembly connection may be designed in such a way that the connection element, in respect of its longitudinal direction, is connected to the holding assembly in such a way that it can be displaced relative to the holding assembly at least within a predetermined range. The holding assembly connection preferably comprises an eyelet or is preferably designed as an eyelet the connection element extends through, i.e. that it runs through. For example, the holding assembly connection may optionally be designed as a strap attached to the holding assembly by both ends, thus forming a loop or the eyelet, respectively. This allows the connection element to move about relative to the holding assembly.

According to a further preferred embodiment, the tightening assembly comprises a deflection eyelet through which the connection element extends at the first connection or the second connection, wherein the connection element comprises a fastening section with respect to the deflection eyelet opposite the holding assembly connection for fastening the connection element to the support assembly.

In the case in hand, an “eyelet” is generally understood to be an opening the connection element may be passed through, or the connection element may extend through. Preferably, the dimensions of the eyelet are equal to or greater than the dimensions of the cross-section of the connection element, and/or the eyelet can be expanded, in other words stretched, in any case at least to the dimensions of the cross-section of the connection element. An “eyelet” may be closed or open, i.e. the eyelet may have a closed contour, corresponding to a closed ring or hole. Alternatively, the contour may be interrupted, in the sense of a slot or a groove. An open eyelet may therefore correspond to a hook. The term “eyelet” or “deflection eyelet” generally includes an opening, for example provided in the form of a ring, a loop, a hole or a hook.

The deflection eyelet or the connection it is comprised in is attached to the support assembly at a predetermined position. Accordingly, the connection element between the holding assembly connection and the connection comprising the deflection eyelet is oriented in a predetermined direction in the tightened state, so that the direction of force transmission is affected as intended. This is essentially independent of the length of the connection element between the connection comprising the deflection eyelet and the holding assembly connection. Without this being mandatory, the connection element can or may be deflected via the deflection eyelet so as to be fastened, preferably releasably, to a pre-determined area on the support assembly.

According to a further preferred embodiment, from a medial-lateral point of view, the fastening section can be detachably fastened to an adhesive section of the support assembly on the side opposite the deflection eyelet. As a result, the support assembly may optionally be tightened, in particular if it extends from the connection comprising the deflection eyelet to the adhesive section from a posterior direction and with a substantially tensile-rigid material.

According to a further preferred embodiment, the support assembly comprises a strap for fixing the support array to the lower leg and/or proximally to the ankle joint, which, preferably medial-laterally from the opposite side of the deflection eyelet, extends from the support assembly and at its free end is provided with a fastening area that can be fastened to a fastening area of the support assembly and preferably can be attached on the side of the deflection eyelet to an adhesive area of the support assembly from a medial-lateral point of view. Preferably, it is possible to derive an area of the support assembly characterised by tensile stiffness due to the detachable fastening of the strap to the adhesion area in respect of the longitudinal axis of the lower leg or in respect of a proximal-distal direction in the circumferential direction. The tensile-stiff area in any case extends in the circumferential direction from the base of the strap on the support assembly to the adhesive area.

Preferably, the fastening area and the adhesive area are designed as a velcro fastener.

If the strap and the fastening section of the connection element are optionally arranged such on the support assembly that they can be tightened in essentially opposite directions, the strap and the fastening section of the connection element can be pulled in opposite directions at the same time. On the one hand, this tightens the support assembly and fixes it to the lower leg and/or the proximal area of the ankle and, on the other hand, this simultaneously tightens the connection element or conveys it into the tightened state, respectively. This optional embodiment therefore in any case allows for the device to be attached to the wearer's foot particularly quickly and ergonomically. Due to the tightening process in opposite directions, the support assembly can also be prevented from twisting in relation to a longitudinal axis of the lower leg. Due to the tightening at the connection element, force is introduced to the support assembly that counteracts the force introduced by tightening the strap. The resulting torques applied to the support assembly in respect of the longitudinal axis of the lower leg as the axis of rotation therefore essentially cancel each other out.

Preferably, the strap and the fastening section are arranged in such a way that they overlap in the tightened state, preferably overlapping essentially in the circumferential direction. In other words, according to a preferred embodiment, as per the radial direction, one of the strap[s] and the fastening section[s] can be arranged above the other of the strap[s] and the fastening section[s] in the tightened state, where the device is fastened to and closed on the wearer's foot, i.e. the strap is arranged above the fastening section or vice versa. The holding forces generated when the device is worn are therefore essentially in a single line of force. Consequently, they cause essentially no moments [of force] otherwise acting on the support assembly radially to the longitudinal axis of the lower leg that could potentially cause displacement and/or rotation of the support assembly at the lower extremity of the wearer due to the differing lines of force.

According to a further preferred embodiment, the strap comprises an opening through which the fastening section of the connection element can be inserted, the opening preferably being arranged between the adhesive section and the deflection eyelet on the support assembly. As a result, the strap and the fastening section may extend in opposite directions and at the same height in the circumferential direction in respect of the longitudinal axis of the lower leg. As a result, they may overlap in opposite directions, with the strap on one side of the opening being arranged above the connection element as per the radial direction and the strap on the other side of the opening being arranged below the connection element. The tensile forces introduced during tightening and the holding forces generated when the device is worn are therefore essentially in a single line of force. Consequently, they essentially do not cause any displacement and/or rotation in respect of the support assembly that would otherwise be generated radially to the longitudinal axis of the lower leg due to moments of force resulting from different lines of force.

According to a further preferred embodiment, the tightening assembly comprises a rotary knob roll-up lacing system. The design of rotary knob roll-up lacing systems is generally known in the art and can be found, for example, in EP 2 378 911 A2 or US 2017/0325548 A1.

Alternatively, or additionally, the tightening assembly may comprise a plurality of hooks arranged consecutively on the support assembly in line with the direction of the alignment of the hooks and a fixing hook arranged on the connection element for hooking into one of the plurality of hooks on the support element—or vice versa.

Furthermore, the support assembly may optionally comprise a clamping unit for clamping the connection element, preferably a cord stopper. Preferably, the clamping unit is attached to the support assembly. It is preferably designed as a detachable clamping unit, so that the clamping of the connection element in the clamping unit can be released. In the optional embodiment as a cord stopper, which is also referred to synonymously as a tanka or cord clamp, the clamping may, for example, be spring-loaded, so that the clamping can be released by pressing a button to release the pre-tension.

According to a further preferred embodiment, the connection element comprises a damping element or the connection element is connected to the lateral connection via a damping element, or to the medial connection or to the holding assembly connection.

A “damping element” is understood to be a device that provides damping of a relative movement between two components of the damping element as a function of speed or acceleration. The damping element therefore damps a relative movement between the support assembly and the holding assembly as a function of speed or acceleration.

The damping element is preferably provided as an adaptive damping element. In other words, the damping element has a preferably low first damping constant below a predetermined threshold limit speed or threshold limit acceleration and a preferably higher damping constant starting at or above the predetermined threshold limit speed or threshold limit acceleration. This allows for the damping element to provide a lower damping effect below the specified threshold limit speed or threshold limit acceleration in accordance with the ratio of the damping constants than above the threshold limit speed or threshold limit acceleration. It has been shown that this allows for providing almost unhindered mobility of the ankle joint, particularly for movements at moderate speeds or accelerations. If inversion occurs at high speeds and/or accelerations where there is a risk of injury to the ligaments of the ankle joint, the foot movement damping device dampens the inversion movement via the damping element and injury is avoided.

According to another preferred embodiment, the tightening assembly is configured in such a way that the damping element can be conveyed into a predetermined state, preferably comprising a predetermined length and/or a predetermined pre-tension, by tightening the connection element.

According to another preferred embodiment, the connection element is releasably fastened to the support assembly by a free end, wherein preferably the fastening of the free end to the support assembly at least partially forms the first connection or the second connection, wherein preferably a fastening section of the connection element is provided at the free end, which is detachably fastened to an adhesive section of the support assembly, wherein the fastening section and the adhesive section are preferably designed as a velcro fastener. As a result, the device can be implemented in a particularly compact fashion, and particularly favourable force transmission into the lower leg can be achieved.

The above object is further solved by a shoe for damping and/or limiting a foot movement via the ankle joint employing the features of claim 11. Advantageous further embodiments of the shoe are shown in the dependent claims, the description in hand and the figures.

Accordingly, a shoe is suggested for damping and/or limiting a foot movement via the ankle joint, comprising a sole area and an upper shoe area connected to the sole area. The shoe, moreover, comprises a device in accordance with any of the above embodiments.

The shoe is capable of achieving the advantages and effects described in connection with the device in an analogous fashion.

According to a preferred embodiment, the sole area of the shoe comprises the holding assembly, preferably with the holding assembly being integrated into the sole area, and the shoe top, preferably an upper portion of the shoe top, comprises the support assembly, wherein preferably the support assembly is integrated into the upper area of the shoe top, wherein preferably the device is essentially completely integrated into the shoe.

### BRIEF DESCRIPTION OF THE DRAWINGS

Preferred embodiments of the invention are illustrated in further detail by the subsequent description of the Figures. In this:

FIG. 1 schematically shows a perspective lateral view of a device for limiting and/or damping a foot movement via the ankle joint;

FIG. 2 schematically shows a perspective lateral view of a further device for limiting and/or damping a foot movement via the ankle joint;

FIG. 3 schematically shows a perspective lateral view of a further device for limiting and/or damping a foot movement via the ankle joint;

FIG. 4 schematically shows a perspective lateral view of a further device for limiting and/or damping a foot movement via the ankle joint;

FIG. 5 schematically shows a perspective lateral view of a further device for limiting and/or damping a foot movement via the ankle joint;

FIG. 6 schematically shows a perspective lateral view of a further device for limiting and/or damping a foot movement via the ankle joint;

FIG. 7 schematically shows a perspective lateral view of a further device for limiting and/or damping a foot movement via the ankle joint;

FIG. 8 schematically shows a perspective lateral view of a further device for limiting and/or damping a foot movement via the ankle joint;

FIG. 9 schematically shows a perspective lateral view of a further device for limiting and/or damping a foot movement via the ankle joint;

FIG. 10 schematically shows a perspective lateral view of a further device for limiting and/or damping a foot movement via the ankle joint;

FIG. 11 schematically shows a medial lateral view of the device of FIG. 10;

FIG. 12 schematically shows a lateral side view of the device of FIG. 10;

FIG. 13 schematically shows a further perspective lateral view of the device of FIG. 10;

FIG. 14 schematically shows a further perspective lateral view of the device of FIG. 10;

FIG. 15 schematically shows a perspective lateral view of a further device for limiting and/or damping a foot movement via the ankle joint;

FIG. 16 schematically shows a sectional view of the device of FIG. 15 through its support assembly perpendicular to the proximal distal direction; and

FIG. 17 schematically shows a sectional view of a further device for limiting and/or damping a foot movement via the ankle joint through its support assembly perpendicular to the proximal distal direction.

### DETAILED DESCRIPTION

Described hereinafter are preferred embodiments based on the Figures. In this, identical or similar elements or elements with the same effect are referenced with identical reference numerals in the different Figures, and these elements are not repeatedly described so as to avoid redundancies.

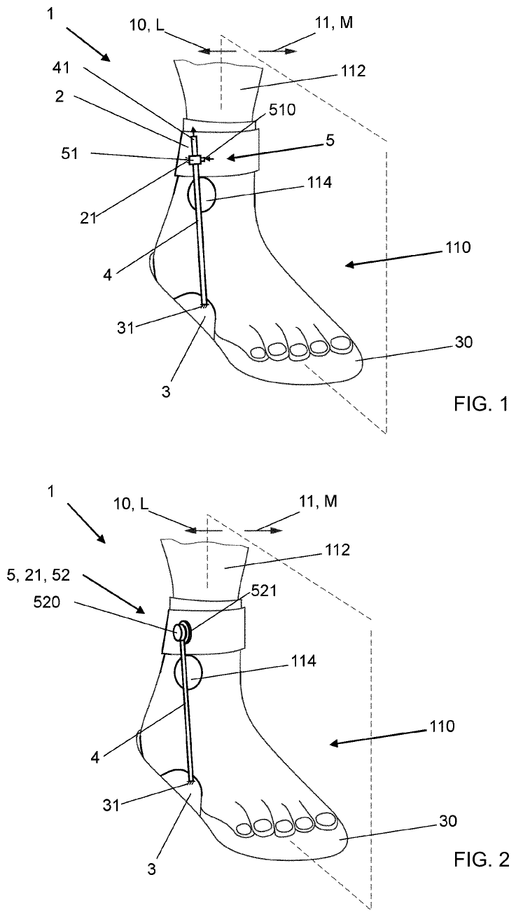

FIG. 1 schematically shows a perspective side view of a device 1 for limiting a foot movement via the ankle joint, which is attached to a human foot 110. Device 1 is attached to the foot 110 below the ankle 114, the malleolus fork, the ankle joint of the foot 110 via a holding assembly 3. Above the ankle 114, the foot movement damping device 1 is fastened to a lower leg 112 by means of a support assembly 2.

To enable connection to the foot 110, the holding assembly 3 is equipped with a sole 30 for the sole of the foot 110 to rest on. Sole 30 is provided with a holding assembly connection 31 on a first side 10 of the foot movement damping device 1, which in the case in hand corresponds to the lateral side L of the foot 110.

Device 1 further comprises a connection element 4, which is provided in the form of a strap that is characterised by tensile stiffness in the longitudinal extension of the strap and extends between the support assembly 2 and the holding assembly 3 and is connected to each of these. The connection element 4 is connected to the holding assembly 3 via the holding assembly connection 31, in the case in hand optionally sewn, welded and/or glued. The connection element 4 is connected to the support assembly 2 via a first connection 21 arranged on the lateral side L. The first connection 21 is therefore a lateral connection.

The device 1 further comprises a tightening assembly 5 for tightening the connection element 4, and the tightening assembly is designed such that the connection element 4 between the holding assembly 3 and the support assembly 2 can be switched from a loosened state to a tightened state and vice versa. In the case in hand, the tightening assembly 5 comprises a clamping unit for clamping the connection element, which is presently designed as a cord stopper 51. A clamping force applied to the connection element 4 via the cord stopper 51 prevents the connection element 4 from slipping within the cord stopper 51. The cord stopper 51 is fixed to the support assembly 2 and thus forms the first connection 21. By pulling the free end 41 of the connection element 2 in a substantially proximal direction, as indicated by the arrow at the end 41, the connection element 4 can be conveyed into a tightened state between the first connection 21 and the holding assembly connection 31, i.e. between the support assembly 2 and the holding assembly 3, where it is then provided at a predetermined pre-tension. A button 510 of the cord stopper 51 can be pressed in order to release the tightened state, by way of releasing the clamping of the connection element 4 in the cord stopper 51. The device 1 is configured in such a way that the tightened state of the connection element 4 can be set in a neutral position of the foot.

FIG. 2 schematically shows a perspective side view of a further device 1 for limiting a foot movement via the ankle joint, which essentially corresponds to that of FIG. 1, wherein the tightening assembly comprises a rotary knob roll-up lacing system 52 instead of the cord stopper 51 (see FIG. 1), which is attached to the support assembly 2 by its base 521 and which is connected to the proximal end of the connection element 4. By turning the rotary knob 520 of the rotary knob roll-up lacing system 52, clockwise in the case in hand, the connection element 4 is rolled up in the rotary knob roll-up lacing system 52, so that the connection element 4 can be conveyed into the tightened state. By lifting the rotary knob 520, the rotary knob roll-up lacing system 52 is switched to free rotation and the connection element 4 is conveyed into the released state, accordingly.

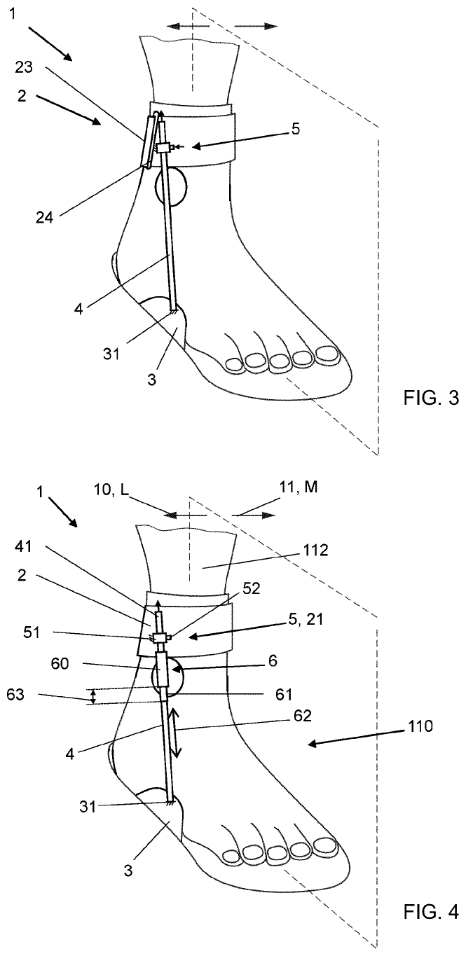

FIG. 3 schematically shows a perspective side view of a further device 1 for limiting a foot movement via the ankle joint, which essentially corresponds to the device shown in FIG. 1. It moreover comprises a strap 23, which runs through a strap eyelet 24 on the support assembly 2 and is deflected on said eyelet. The strap 23 is provided as a velcro strap and can be pulled tight at the deflection eyelet and, on the posterior side of the support assembly 2, engage with a velcro area on the support assembly 2 by its free end (not shown) that is equipped with a velcro area so as to form a velcro fastener. The support assembly 2 can thus be fixed in the intended position above the malleolus fork.

FIG. 4 schematically shows a perspective side view of a further device 1 for limiting and/or damping a foot movement via the ankle joint. It also comprises a damping element 6, which is integrated into the connection element 4 between the first connection 21 and the holding assembly connection 31. The design of the damping element 6 derives from DE 10 2018 131 457 A1, and we incorporate this design by way of reference. The damping element 6 comprises a receptacle 60, in which a force transmission body 61 is held movably relative to the receptacle 60 in the longitudinal direction 62. In other words, the force transmission body 61 can telescope out of the receptacle 60 and into the receptacle 60. The damping element 6 is provided with a reset unit, not shown here, for resetting the damping element 6 to a retracted position.

The tightening assembly 5 is configured such that by tightening the connection element 4 as described with respect to FIG. 1, the damping element 6 can be conveyed into a predetermined state, preferably comprising a predetermined length and/or a predetermined pre-tension, in the tightened state of the connection element 4. The tightened state of the connection element 4 manifests when the force transmission body 61 protrudes from the receptacle 60 by a predetermined length 63. The length 63 may be indicated by a marking on the force transmission body 61.

Accordingly, the support assembly 2 and the holding assembly 3 are connected via the connection element 4 comprising the damping element 6. The damping element 4 is designed as an adaptive damping element 6, so that a relative movement between the support assembly 2 and the holding assembly 3 is damped adaptively, presently as a function of the movement's respective speed and acceleration.

If the ankle joint is subjected to inversion, the distance between the holding assembly 3 and the support assembly 2 increases on the first, lateral side 10, L. Consequently, the distance between the first connection 21 and the holding assembly connection 31 increases. Accordingly, the length of the connection element 4 would also have to increase in order to allow for an inversion movement. In order to generally enable such a movement within the physiological range, the damping element 6 is provided. During movements at moderate speeds or accelerations, the ankle joint can move almost unhindered. If inversion occurs at high speeds and/or accelerations where there is a risk of injury to the ligaments of the ankle joint, the damping element 6 dampens the inversion movement and injury is avoided.

Since in the present context the tightened state of connection element 4 is pre-set for the neutral position of the foot, damping element 6 or rather its effect described above only becomes effective starting at inversion movements exceeding the neutral position (inversion angle of 0°) in the direction of inversion.

The device 1 shown in FIG. 1 is designed as a completely stand-alone device 1, which exhibits its protective effect due to its structure. It is not necessary for a person wearing the device 1 to put on a shoe over the device 1 in order to achieve the aforementioned protective effect. However, the device 1 can be designed in such a way that it is possible to wear the device 1 under or in a shoe without causing pressure marks on the foot. For this purpose, the sole 30 may preferably be designed as an insole, to replace an insole in a shoe or that could be placed on top of an insole or underneath an insole. Alternatively, the support assembly 2 and the holding assembly 3 can be arranged on a bandage or a basic structure, such as a stocking made of an elastic compression knit fabric, which is capable of adapting to the wearer's body.

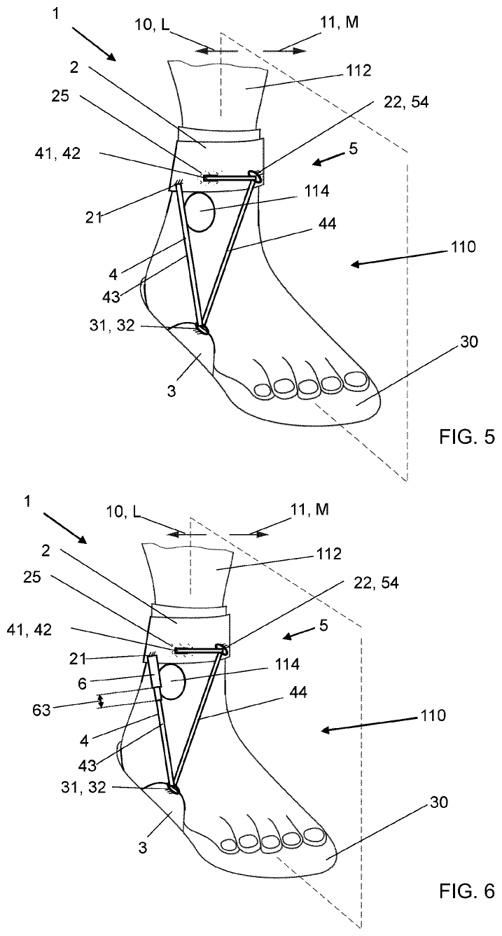

FIG. 5 schematically shows a perspective side view of a further device 1 for limiting a foot movement via the ankle joint, which essentially corresponds to the device shown in FIG. 1. In contrast to the devices 1 shown in FIGS. 1 to 4, the device 1 in FIG. 5 comprises a connection element 4, which is fastened to the support assembly by a lateral first connection 21 and a medial second connection 22. Between the first and second connections 21, 22, the connection element 4 is connected to the holding assembly 3 by the holding assembly connection 31 on the lateral side L. The connection element 4 is thus divided into a first arm 43 extending between the first connection 21 and the holding assembly connection 31 and a second arm 44 extending between the holding assembly connection 31 and the second connection 21.

The connection element 4 is fastened to the holding assembly connection 31 so as to be displaceable relative to the holding assembly 3. For this purpose, the connection element 4, which is designed as a strap characterised by tensile stiffness, is moveably threaded through an eyelet 32, which in turn forms the holding assembly connection 31, and is thus guided therein.

In the event of plantar flexion or dorsiflexion at the ankle joint (or at its upper ankle joint axis), the point at which the connection element 4 is deflected in the eyelet 32 shifts, namely in accordance with the extent of the movement. As a result, the lengths of the first arm 43 and the second arm 44 change slightly. This slight change is compensated for by subtle sliding of the connection element 4 in the eyelet 32, so that any folds or pressure marks on the skin are avoided that could be caused by a rigid connection.

To prevent the support assembly 2 from shifting when force is applied as a result of inversion, the first connection 21 is arranged on the lateral side L and the second connection 22 is arranged on the medial side M. Forces introduced into the support assembly by the connections 21, 22 via the connection element 4 are consequently introduced into the lower leg 112 and/or the proximal part of the ankle 114, in the sense of a linear load extending posteriorly and around between the connections along the support assembly 2.

In this embodiment, the tightening assembly 5 comprises a deflection eyelet 54 through which the connection element 4 extends at the medial second connection 22 and is deflected back towards the lateral side L in the tightened state. In respect of the deflection eyelet 54, the connection element 4 comprises a fastening section 42 opposite the holding assembly connection 31 for fastening the free end 41 of the connection element 4 to an adhesive section 25 of the support assembly 2. In the case in hand, the fastening section 42 and the adhesive section 25 are designed as a velcro fastener

By tightening the fastening element 4 by pulling on its fastening section 42, deflecting it via the deflection eyelet 54 and fastening the fastening section 42 laterally to the deflection eyelet 54, the fastening element can firstly be placed in the tightened state. Secondly, a fastening of the support assembly 2 can be thus achieved to the lower leg 112 and/or ankle 114.

FIG. 6 schematically shows a perspective side view of a further device 1 for limiting and/or damping a foot movement via the ankle joint, which essentially corresponds to the device shown in FIG. 1. In the case in hand, the connection element 4 is connected to the lateral first connection 21 via damping element 6. The damping element 6 essentially corresponds to the damping element described in FIG. 4.

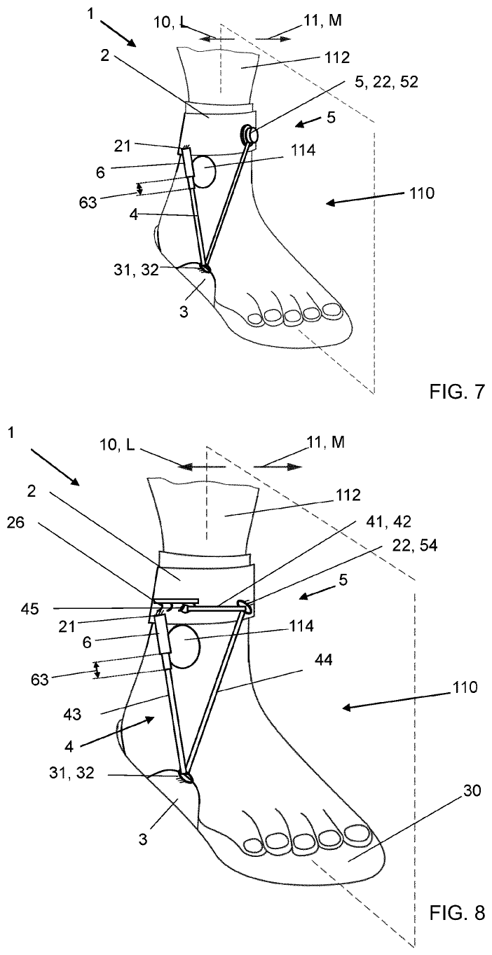

FIG. 7 schematically shows a perspective side view of a further device 1 for limiting and/or damping a foot movement via the ankle joint, which essentially corresponds to the device shown in FIG. 6. However, in the case in hand, the tightening assembly 5 is designed in analogy to FIG. 2 in the form of a rotary knob roll-up lacing system 52 arranged on the medial second connection 22.

Alternatively, a clamping unit, for example in analogy to FIG. 1, can be provided on the first and/or second connection 21, 22 instead of the rotary knob roll-up lacing system 52.

FIG. 8 schematically shows a perspective lateral view of a further device 1 for limiting and/or damping a foot movement via the ankle joint, which essentially corresponds to that in FIG. 6, wherein instead of the velcro fastener consisting of fastening section 42 and adhesive section 25 as shown in FIG. 6, a plurality of hooks 26 arranged consecutively on the support assembly 2 in line with the direction of the alignment of the hooks and a fixing hook 45 arranged at the free end 41 of the connection element 4 for hooking into one of the plurality of hooks 26 are formed as part of the tightening assembly 5.

Alternatively, instead of the velcro fastener, a rotary knob roll-up lacing system 52 or a clamping unit may be provided, by means of which the connection element 4 deflected in the deflection eyelet 54 can be tightened.

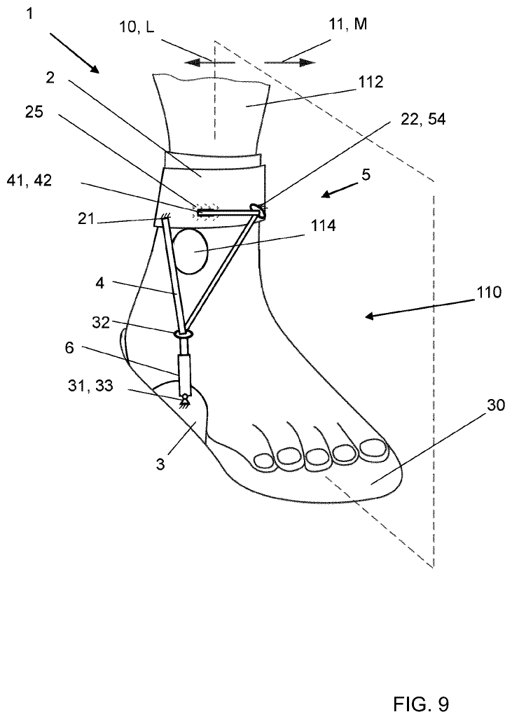

FIG. 9 is a schematic lateral side view of a further device 1 for limiting and/or damping a foot movement via the ankle joint, which essentially corresponds to the device shown in FIG. 5, whereas in this embodiment the holding assembly connection 31 comprises the damping element 6 corresponding to FIG. 4 or 6. Similar to what is shown in FIGS. 5 to 8, the connection element 4 is connected to an eyelet 32 of the holding assembly connection 31 so that it is displaceably held relative to the holding assembly connection 31. In other words, the connection element 4 is connected to the holding assembly 3 or the holding assembly connection 31 via the damping element 6.

As indicated by reference numeral 33, the damping element 4 is connected to the holding assembly 3 in an articulated manner.

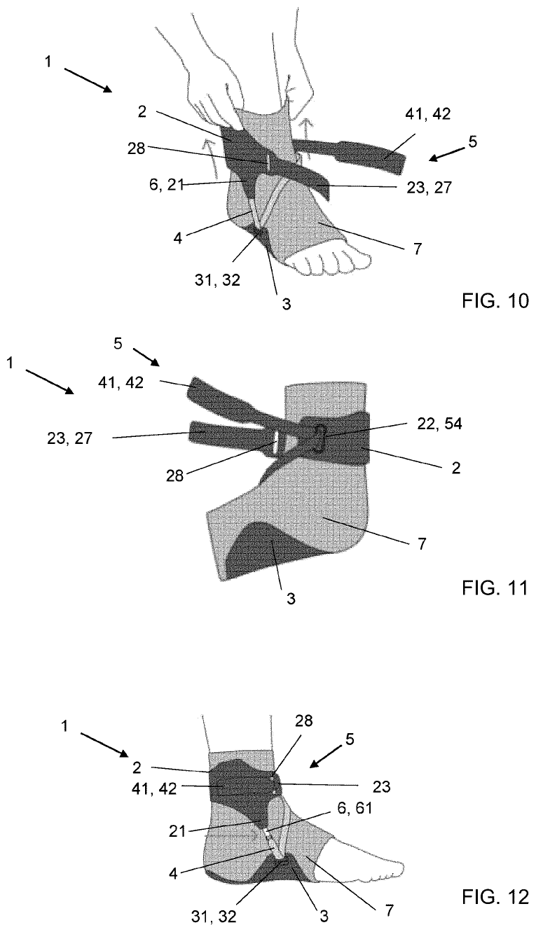

FIG. 10 schematically shows a perspective side view of a further device 1 for limiting and/or damping a foot movement via the ankle joint, and FIG. 11 shows the device 1 of FIG. 10 in a medial side view. The structure of the device 1 according to these Figures essentially corresponds to the embodiment shown in FIG. 6. In the embodiment shown in FIGS. 10 and 11, the basic structure of the device 1 comprises a stocking 7 or a bandage made of a knitted elastic compression fabric, which due to its elasticity, is capable of adapting to the wearer's body. The holding assembly 3 is attached to the underside of the stocking 7, essentially in the sole area. The support assembly 2 is attached to the stocking 7 essentially proximally and in the area of the ankle 114.

On the lateral side, as in FIG. 6, the device 1 comprises the damping element 6, which is integrated into the support structure 2 and is therefore essentially invisible from the outside. On one side, the damping element 6 is connected to the support structure 2, and on the other side, the damping element 6 is connected to the connection element 4. The damping element 6 can therefore be regarded as part of the lateral, first connection 21.

In analogy to FIG. 6, the connection element 4 extends from the lateral, first connection 21 to the holding assembly 3, is deflected there in an eyelet 32 serving as a holding assembly connection 31 and extends further from the holding assembly connection 31 to the, preferably medial, second connection 22, which comprises the deflection eyelet 54. Therein, the connection element 4 is deflected again, namely in order to be releasably fastened to the fastening section 42 on the lateral side L at the support assembly 2, when the device 1 is in a closed state, and in the case in hand this is achieved via the velcro fastener already described with respect to FIG. 6.

The support assembly 2 further comprises a strap 23 for securing the support assembly 2 to the lower leg 112 and/or proximal to the ankle joint ankle 114, which extends from the support assembly 2 on the lateral side L of the support assembly 2 and includes a fastening area 27 at its free end, which can be fastened to an adhesive section (not shown) of the support assembly 2 arranged on the medial side M. The fastening area 27 is designed as a velcro strip, which may create a hook-and-loop fastening with the material of the support assembly 2.

The strap 23 and the fastening section 42 are arranged on the support assembly 2 in such a way that they can be tightened in essentially opposite directions. For this purpose, the strap 23 or the support assembly 2 comprises an opening 28 at the base of the strap 23, through which the fastening section 42 of the connection element 4 can be inserted.

In FIGS. 10 and 11, the device 1 is shown in a released state of the connection element 4. In this state, the free end 41 of the connection element 4 is optionally not threaded through the opening 28. As the free end 41 is released from the support assembly 2, the connection element 4 can slide through the deflection eyelet 54 and the eyelet 32 in the direction of the longitudinal extension of the connection element 4. Consequently, when the device 1 is put on the wearer's foot, as indicated in FIG. 10 by the hands and the arrows laterally and medially of the device 1, expansion of the device 1 or the stocking 7 is not limited by the connection element 4.

FIG. 12 schematically shows a lateral side view of the device 1 of FIG. 10, with the connection element 4 being shown in the tightened state. The free end 41 or the fastening section 42 is releasably fastened to the lateral side L at the support assembly 2. In the tightened state, the connection element 4 is in the neutral position of the foot as shown in FIG. 12, indicating pretension in the longitudinal direction, in a predetermined amount. In the case in hand, the amount of pretension is such that the side of the damping element 6 on the connection element side, which presently corresponds to the force transmission body 61, is pulled out of the support assembly 2 by a predetermined length (or amount), here optionally 5-10 mm, against the resetting force provided by the resetting unit of the damping element 6, which compresses the damping element 6 into a compressed position.

Returning from the tightened state to the released state of the connection element 4 can be affected by releasing the fastening section 42 of the support assembly 2. Accordingly, the tightening assembly comprises at least the eyelet 32, the deflection eyelet 54 and the fastening section 42.

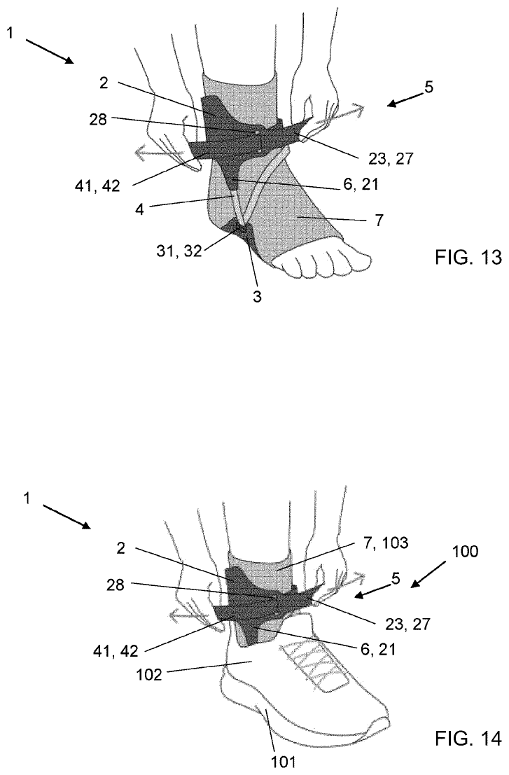

FIG. 13 schematically shows a further perspective side view of the device 1 of FIG. 10 during a process of tightening the connection element 4 by actuating the tightening assembly 5. In the case in hand, tightening of the connection element 4 is affected by pulling on the strap 23 and the free end 41 of the connection element 4 in essentially opposite directions, in the present case in an essentially lateral and an essentially medial direction, and the strap 23 and the free end 41 are fastened to the support assembly 2 while being kept under tension.

FIG. 14 schematically shows a further perspective lateral view of the device 1 of FIG. 10. In this Figure, the wearer has also put on a shoe 100, which they have pulled over the device 1. As again indicated by the arrows shown in FIG. 14, the connection element 4 can be re-tensioned or re-tightened by releasing the free end 41 and the strap 23 from the support assembly 2 and pulling on them again as described in connection with FIG. 13.

Alternatively, the device 1 can also be designed as part of the shoe 100 and accordingly be at least partially integrated into it and/or permanently connected to it. For example, the stocking 7 can be designed as a component of the shoe 100.

Alternatively, or additionally, the sole area 101 of the shoe 100 can comprise the holding assembly 3, which is then preferably integrated into the sole area 101. Furthermore, the upper part of the shoe 102 of the shoe 100 may comprise the support assembly 2, preferably in an upper area 103 of the shoe 100, which can be designed as a stocking 7, for example.

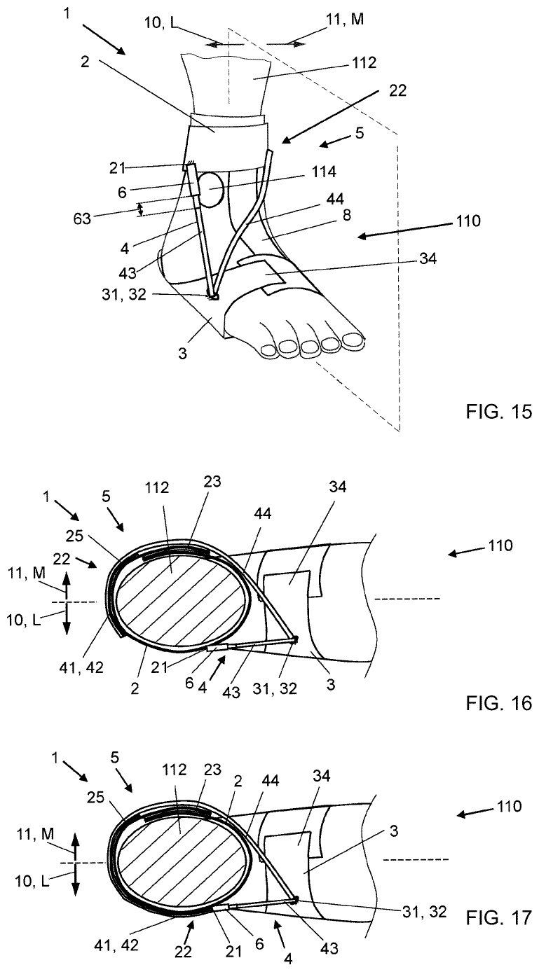

FIG. 15 shows a schematic perspective lateral view of a further device 1 for limiting and/or damping a foot movement via the ankle joint. The basic structure of the device 1 essentially corresponds to the structure shown in FIG. 6. In contrast to the embodiment in FIG. 6, the embodiment according to FIG. 15 comprises a holding assembly 3, which can be releasably closed by a velcro strap 34 in the circumferential direction of the foot 110.

In addition, the support assembly 2 and the holding assembly 3 are connected to one another via a preferably tension-resistant connection segment 8.

In analogy to FIG. 6, the connection element 4 is connected to the support assembly 2 on the lateral side L at the first connection 21 via the damping element 6 and extends from the first connection 21 to the holding assembly connection 31, at which it is displaceably held in the eyelet 32.

In contrast to the embodiment shown in FIG. 6, the second arm 44 of the connection element 4 does not extend through a deflection eyelet and anteriorly back to the lateral side L. In this case, the connection element 4 extends from the holding assembly connection 31 (anteriorly) to the medial side M to the support assembly 2 and is detachably fastened to the latter via the fastening section 42, provided in the form of a velcro section on the adhesive section 25 of the support assembly 2.

The alignment of the second arm 44 up to the free end 41 of the connection element 4 derives from FIG. 16, which schematically shows a sectional view of the device of FIG. 15 through its support assembly 2 perpendicular to the proximal-distal direction above the ankle 114. As can be seen from this Figure, the fastening section 42 and the adhesive section 25 are optionally arranged at least partially medially on the posterior side. The fastening section 42 and the adhesive section 25 have corresponding hook-and-loop areas, which together form a velcro fastening when connected to one another, so that the releasable fastening of the free end 41 of the connection element 4 is created. The connection element 4 is thus supported on the medial side M via the second arm 44 and is supported on the posterior side via the support assembly 2 and thus via the lower leg 110 and/or ankle 114 of the wearer of the device 1 and is fixed in respect of its position relative to the support assembly 2 by the velcro fastening. Pursuant to this embodiment, this accordingly represents the second connection 22.

As can be seen from FIG. 16, the support assembly comprises a strap 23 for fastening the support assembly 2 to the lower leg 112 and/or proximal to the ankle joint 114. According to this optional embodiment, the strap 23 and the connection element 4 are arranged such that they overlap in the tightened state, as shown in FIG. 16, in case in hand essentially in the circumferential direction with respect to a proximal-distal oriented axis. In respect of the proximal-distal direction or axis, the connection element 4 is arranged radially outside, i.e. above the strap 23. The connection element 4 thus overlaps with strap 23.

FIG. 17 schematically shows a sectional view of through a further device 1 for limiting and/or damping a foot movement via the ankle joint through its support assembly 2 perpendicular to the proximal distal direction. The device 1 essentially corresponds to the device shown in FIGS. 15 and 16, whereas here the fastening section 42 and the adhesive section 25 extend to the area of the first connection 21 on the lateral side L. The connection element 4 thus extends from the first connection 21 arranged on the lateral side L to the holding assembly connection 31, where it is moveably held, and from there it further extends anteriorly around to the medial side M, and from there it further extends again posteriorly around to the lateral side L into the area of the first connection 21. As a result, a frictional connection can be effected between the first connection 21 and the free end 41 of the connection element 4 on the lateral side L if the free end 41 or the fastening section 42 are detachably fastened to the support assembly 2 or the adhesive section 25. The connection element 4 then accordingly forms a loop running from the lateral side L via the shoe heel area and posteriorly around to the medial side M, and transfers tensile forces introduced at the holding assembly connection 3 at the support assembly 2 in the shoe heel area as well as on the medial side M and partially also on the lateral side L into the support assembly 2, and from there further onto the lower leg 110 and/or ankle 114 of the wearer of the device 1.

Optionally, the second arm 44 may extend through a preferably open deflection eyelet on the medial side M. In other words, the second arm 44 may be hooked into the open deflection eyelet in the released state to provide a predetermined path on the anterior side for the second arm 44 from the support assembly 3 to the deflection eyelet. The deflection eyelet can be considered as the start of the second connection 22, and the end of the second connection 22 can correspondingly considered as the free end 41.

Alternatively, the connection element 4 may also not comprise a damping element 6, but instead be connected directly to the support assembly 2 via the first connection 21, for example, as shown in FIG. 5.

Insofar as applicable, all individual features illustrated in the sample embodiments can be combined and/or exchanged without leaving the scope of the invention.

1. A device (1) for limiting and/or damping a foot movement via the ankle joint, the device comprising: a support assembly (2) for support on a lower limb (112) and/or proximally on the ankle-joint malleolus (114); a holding assembly (3) for holding on a foot (110), preferably on a sole region of a foot (110); and at least one connection element (4), which extends between the support assembly (2) and the holding assembly (3) and is connected to each, characterised by
a tightening assembly (5) for tightening the connection element (4), which tightening assembly is designed such that the connection element (4) between the holding assembly (3) and the support assembly (2) can be switched from a loosened state to a tightened state and vice versa.

2. The device (1) according to claim 1, characterised in that the connection element (4) is connected to a first connection (21) and a second connection (22) on the support assembly (2) and is connected between the first and second connections (21, 22) to a holding assembly connection (31) on the holding assembly (3), wherein preferably the tightening assembly (5) is configured for tightening the connection element (4) between the first connection (21) and the second connection (22), and/or the first connection (21) is a lateral connection (21) and the second connection (22) is a medial connection (22), and/or the connection element (4) is held on the holding assembly connection (31) so as to be displaceable relative to the holding assembly (3).

3. The device (1) according to claim 2, characterised in that the support assembly (2) comprises the tightening assembly (5) for tightening the connection element (4).

4. The device (1) according to claim 3, characterised in that the tightening assembly (5) comprises a deflection eyelet (54) through which the connection element (4) extends at the first connection (21) or the second connection (22), wherein the connection element (4) comprises a fastening section (42) with respect to the deflection eyelet (54) opposite the holding assembly connection (31) for preferably releasably fastening the connection element (4) to the support assembly (2).

5. The device (1) according to claim 4, characterised in that the fastening section (42) can be detachably fastened to an adhesive section (25) of the support assembly (2) on the side (L, M) opposite the deflection eyelet (54), from a medial lateral perspective.

6. The device (1) according to claim 5, characterised in that the support assembly (2) comprises a strap (23) for fixing the support assembly (2) to the lower leg (112) and/or proximally to the ankle joint (114).

7. The device (1) according to claim 6, characterised in that the support assembly (2) extends away from the support assembly (2), preferably medial-laterally from the opposite side (L, M) of the deflection eyelet (54), and that at its free end is provided with a fastening area (27) which, preferably medial-laterally on the side (L, M) of the deflection eyelet (54), can be attached to an adhesive area of the support assembly (2), wherein preferably the fastening section (42) and the adhesive section (25) and/or the fastening area (27) and the adhesive area are designed as a velcro fastener, wherein preferably the strap (23) and the fastening section (42) are arranged such on the support assembly (2) that they are tightened in essentially opposite directions.

8. The device (1) according to claim 7, characterised in that the strap (23) comprises an opening (28) through which the fastening section (42) of the connection element (4) can be inserted, with the opening (28) preferably being arranged between the adhesive section (25) and the deflection eyelet (54).

9. The device (1) according to claim 8, characterised in that the tightening assembly (5) comprises a rotary knob roll-up lacing system (52), or the tightening assembly (5) comprises a plurality of hooks (26) arranged consecutively in alignment on the support assembly (2) and a fixing hook (45) arranged on the connection element (4) for hooking into one of the plurality of hooks (26) on the support element (2), or that the support assembly (2) comprises a clamping unit for clamping the connection element (4), preferably a cord stopper (51).

10. The device (1) according to claim 9, characterised in that the connection element (4) comprises a damping element (6), or if dependent on claim 2, that the connection element (4) is connected via a damping element (6) to the support assembly (2), preferably to the first connection (21) and/or to the second connection (22), or to the holding assembly (3).

11. The device (1) according to claim 10, characterised in that the tightening assembly (5) is configured in such a way that the damping element (6) can be conveyed into a predetermined state, preferably comprising a predetermined length and/or a predetermined pre-tension, by tightening the connection element (4).

12. The device (1) according to claim 2, characterised in that the connection element (4) can be detachably fastened to the support assembly by a free end (41).

13. The device (1) according to claim 12, characterised in that the fastening of the free end (41) to the support assembly (2) at least partially forms the first connection (21) or the second connection (22), wherein preferably a fastening section (42) of the connection element (4) is provided at the free end (41), which is detachably fastened to an adhesive section (25) of the support assembly, wherein the fastening section (42) and the adhesive section (25) are preferably designed as a Velcro fastener.

14. A shoe (100) for damping and/or limiting a foot movement via the ankle joint, comprising a sole area (101) and shoe top (102) connected to the sole area (101),
characterised by a device (1) according to claim 1.

15. The shoe (100) according to claim 14, wherein the sole area (101) comprises the holding assembly (3), preferably with the holding assembly (3) being integrated into the sole area (101), and the shoe top (102), preferably an upper part (103) of the shoe top (102), comprises the support assembly (2), wherein preferably the support assembly (2) is integrated into the upper part (103) of the shoe top (102), wherein preferably the device (1) is essentially completely integrated into the shoe (100).
